(19) United States
(12) Patent Application Publication
(10) Pub. No.: US 2019/0151352 A1
(43) Pub. Date: May 23, 2019

(54) OPHTHALMIC COMPOSITION COMPRISING A SYNERGISTIC COMBINATION OF GLYCOGEN AND HYALURONIC ACID OR A SALT THEREOF

(71) Applicant: AZIENDE CHIMICHE RIUNITE ANGELINI FRANCESCO A.C.R.A.F. S.p.A., Roma (IT)

(72) Inventors: Elisa LIBERATI, Roma (IT); Vincenzo RUSSO, Aprilia (LT) (IT); Lorella RAGNI, Chiaravalle (AN) (IT); Giuseppe SANTO, Sortino (SR) (IT); Serena TONGIANI, Grottaferrata (Rome) (IT)

(73) Assignee: AZIENDE CHIMICHE RIUNITE ANGELINI FRANCESCO A.C.R.A.F. S.p.A., Roma (IT)

(21) Appl. No.: 16/094,110

(22) PCT Filed: Apr. 28, 2017
(86) PCT No.: PCT/EP2017/060158

§ 371 Date: Oct. 16, 2018

(30) Foreign Application Priority Data
May 5, 2016 (EP) 16168464.2

(51) Int. Cl. A61K 31/728; A61K 31/715; A61K 9/00; A61K 9/08

(52) U.S. Cl. CPC A61K 31/728; A61K 9/08; A61K 9/0048; A61K 31/715

(57) ABSTRACT

This invention relates to an ophthalmic composition comprising a synergistic combination of glycogen and hyaluronic acid or a pharmaceutically acceptable salt thereof, and at least one pharmaceutical acceptable excipient, to a process for the preparation thereof, and to the use thereof for the treatment of dry eye syndrome.

### FIELD OF THE INVENTION

The present invention relates to an ophthalmic composition comprising a synergistic combination of glycogen and hyaluronic acid or a salt thereof, to a process for the preparation thereof, and to the use thereof for the treatment of dry eye syndrome.

In particular, the present invention relates to an ophthalmic composition wherein glycogen and hyaluronic acid (HA) or a pharmaceutically acceptable salt thereof are present in amounts that provide a synergistic increase in therapeutic effectiveness.

The ophthalmic composition of the present invention is useful to relieve the symptoms of the ocular discomfort consequent to chronic lack of sufficient lubrication and moisture of the eye with potential surface epithelial damage.

### STATE OF THE ART

The tear film is a relatively stable, thin film composed of a superficial lipid layer and an aqueous layer intermixed with a mucus gel layer which is partially adherent to the corneal and conjunctiva surface epithelium. Natural tear film is important for the lubrication and maintenance of the eye surface.

Dry eye syndrome (DES) is a multifactorial disease characterized by the inability of the eye to maintain a layer of tears sufficient to lubricate it properly. DES is characterized by a dysfunction of one or more components of the tear film, leading to the loss of tear film stability, to an osmolarity increase of the tear film and inflammation of ocular surface. This condition is associated with symptoms of ocular discomfort such as itchiness, irritation, foreign body sensation, redness, photophobia and pain. These symptoms are often worse toward the end of the day or after prolonged periods of time requiring vision attention such as reading, driving or computer work.

DES can result from one of the following causes: decreased tear production, excessive tear evaporation, an abnormality in the production of mucus or lipids normally found in the tear layer. Poor production of tears by tear glands may be a result of age, hormonal changes, or various autoimmune diseases, such as primary Sjogren syndrome, rheumatoid arthritis, or lupus. Evaporative loss of the watery tear layer is usually a result of an insufficient overlying lipid layer. Some medicaments such as antihistamines, antidepressants, beta-blockers and oral contraceptives may decrease tear production. LASIK and other vision correction procedures can cause dry eye after they penetrate the eye's surface and reduce corneal nerve sensitivity. Afterwards the eye fails to sense the need for lubrication and inadequate tear production results.

DES, if untreated and uncorrected, can result in permanent damage to the eye with degradation of the exposed ocular tissues or a breakdown of the corneal tissue necessitating, in extreme cases, corneal transplants.

The most common treatment for ocular discomfort consequent to chronic lack of sufficient lubrication and moisture of the eye involves the alleviation of the symptoms by topical administration of a tear substitute that adds a volume of liquid to the anterior surface of the eye. Artificial tears try to substitute natural tears mimicking their high content in water and their physio-chemical properties (osmolarity, pH, viscosity, wetting ability). Typical tear substitute compositions comprise water soluble, aqueous polymer compositions. Many polymers have been used in topically administrable ophthalmic compositions. Included among these are cellulosic polymers such as hydroxypropyl methylcellulose, hydroxyethyl cellulose, and ethyl hydroxyethyl cellulose. Also included are synthetic polymers such as carboxyvinyl polymers and polyvinyl alcohol. Still others include polysaccharides such as xanthan gum, guar gum, dextran and hyaluronic acid. Combinations of polymers have also been used in ophthalmic compositions. Certain combinations of polymers are known to provide synergistic effects on viscosity and, in some cases, even a phase transition from a liquid to a gel.

Artificial tears are delivered to the eye as drops and they are subjected to a rapid drainage through the nasolacrimal duct. To overcome this problem the artificial tears are composed of ingredients that increase contact time with the ocular surface. These ingredients are designed to have mucoadhesive properties. One problem is the high viscosity of the ingredients. In many cases, if the composition contains a sufficiently high concentration of the active ingredients, it is so viscous that application is uncomfortable for the patient and the high viscosity leads to problems such as irritation and blurred vision. Various formulation strategies have been implemented in attempts to overcome the disadvantages of the use of highly viscous materials.

One strategy is the use of a less viscous formulation, that relies on its mucoadhesive properties to remain on the surface of the eye. Sodium hyaluronate has mucoadhesive properties, is a viscoelastic polymer and has anti-inflammatory properties, which can be useful in the treatment of the surface inflammation prevalent in DES. It is a high molecular weight polymer and its solutions are highly viscous. Attempts to use HA alone have run into the problem that this ingredient tends to be irritating to the eye when used in concentrations sufficiently high to treat DES.

WO 2009/044423 disclosed ophthalmic solutions indicated for use as tear substitutes, containing a combination of 0.4% of hyaluronic acid and 0.2% of a polysaccharide known as TSP (Tamarindus indica Seed Polysaccharide) which are able, when administered together in a combination, to act synergistically in stimulating the return to normality of the conjunctival mucosa affected by dry eye syndrome.

EP 1069885 disclosed a humectant and lubricant solution for ophthalmic use based on a glycogen polysaccharide, such a solution showing low viscosity and low oncotic pressure and exerting a pleasing refreshing, lubricating and humectant effect on the cornea.

### SUMMARY OF THE INVENTION

The Applicant faced the problem of obtaining an ophthalmic composition for the treatment and/or prevention of DES.

In particular, the Applicant faced the problem of obtaining an ophthalmic composition that is both low-viscous, mucoadhesive and non-irritating, for the treatment of DES.

After extensive investigation, the Applicant has surprisingly found that a composition containing sodium hyaluronate and glycogen shows improved efficacy in reducing inflammatory parameters associated with the symptoms of the ocular discomfort consequent to chronic lack of sufficient lubrication and moisture of the eye, in protecting the eye from an excessive matrix degradation and in promoting corneal re-epithelization consequent to surface epithelial damage, than would be expected from a composition containing an equivalent amount of either component alone, or that would be expected from a combination of the properties of the two components.

The observed synergistic effect between these two ingredients enables formulation of a composition in which they are present in low concentrations, typically in the order of 0.15% for hyaluronic acid (normally as sodium hyaluronate) and 3% for glycogen.

The composition containing the association of sodium hyaluronate and glycogen has the further advantage of being mucoadhesive, pseudoplastic and low viscous.

Accordingly, in a first aspect this invention relates to an ophthalmic composition comprising a synergistic combination of glycogen and hyaluronic acid or a pharmaceutically acceptable salt thereof, and at least one pharmaceutical acceptable excipient, wherein said composition comprises an amount of said glycogen ranging from 1% to 6% w/w and an amount of said hyaluronic acid or a pharmaceutically acceptable salt thereof ranging from 0.05% to 0.3% w/w.

Unless otherwise specified, all percentages w/w (% w/w) are expressed by weight with respect to the total weight of the ophthalmic composition.

In a second aspect, the present invention relates to an ophthalmic composition for use in the treatment of dry eye syndrome comprising a synergistic combination of glycogen and hyaluronic acid or a pharmaceutically acceptable salt thereof, and at least one pharmaceutical acceptable excipient.

According to a further aspect, the present invention also relates to a method for the treatment of dry eye syndrome, wherein the method consists in applying a therapeutically effective amount of an ophthalmic composition comprising a synergistic combination of glycogen and hyaluronic acid or a pharmaceutically acceptable salt thereof, and at least one pharmaceutical acceptable excipient to a patient in need thereof.

### BRIEF DESCRIPTION OF THE FIGURES

FIG. 1 illustrates SEM photographs at magnification of 2,000×, 10,000× and 20,000× of Human Corneal Epithelium (HCE) surfaces after the treatment according to example 3.1b.

FIG. 2 illustrates light microscopy photographs at magnification of 20× of HCE slices after the treatment according to example 3.2b.

FIG. 3 illustrates SEM photographs at magnification of 2,000× of HCE surfaces after the treatment according to example 3.2c.

### DETAILED DESCRIPTION OF THE INVENTION

The ophthalmic composition of the present invention comprises a synergistic combination of glycogen and hyaluronic acid or a pharmaceutically acceptable salt thereof.

The glycogen used in the ophthalmic composition of the present invention is obtained from natural glycogen which may be extracted from animals or vegetables or chemically and enzymatically synthesized. Molluscs, in particular mussels (Mytilus edulis and Mytilus gallus provincialis) are a particularly useful source of glycogen because they are available in large quantities at low cost and contain a reasonable quantity of glycogen (on average between 2.5% and 3.9% by weight). Other natural sources of glycogen include other bivalve molluscs such as clams, oysters, some species of gastropods or sea snails, such as slipper limpets (Crepidula fornicata), as well as organs of vertebrate animals which are rich in glycogen such as the liver and muscles. Another source of glycogen is starch, which can be transformed in glycogen by using specific enzymes (as disclosed in EP1813678).

The glycogen used in the ophthalmic composition of the present invention may be used as such as obtained from the above mentioned extraction processes and chemical or enzymatic synthesis, or may be treated in subsequent purification procedures. The quality of a commercial glycogen depends on the presence of a greater or lesser quantity of protein residues (measured in terms of quantity of nitrogen expressed as ppm) and reducing sugars.

For the purposes of the present invention the use of a glycogen having a low reducing sugars and nitrogen content is preferred. Examples of commercial products preferably used in this invention are glycogens produced and distributed by Sigma-Aldrich.

Preferably, the glycogen used in the present invention comprises less than 1% by weight, and more preferably less than 0.25% by weight of reducing sugars, measured in accordance with the method by F. D. Snell and Snell, “Colorimetric Methods of Analysis”, New York, 1954, vol. III, p. 204.

Preferably the glycogen used in this invention comprises less than 1000 and more preferably less than 100 ppm of nitrogen measured using the Kjeldahl method.

Advantageously the glycogen used in this invention is Polglumyt™ glycogen, the trade name of a deproteinated glycogen produced and distributed by A.C.R.A.F. S.p.A., Rome, Italy, and obtained in accordance with the purification procedure described in patent EP 654048 B1.

The ophthalmic composition of the present invention comprises an amount of glycogen ranging from 1% to 6% w/w, preferably from 2% to 5% w/w, and more preferably from 3% to 4% w/w.

Advantageously, the ophthalmic composition of the present invention comprises an amount of glycogen of about 3% w/w.

The hyaluronic acid is chemically definable as an unbranched glycosaminoglycan, consisting of alternate units of D-glucuronic acid (GlcUA) and N-acetyl-D-glucosamine (GlcNAc) linked via alternating β-1,4 and β-1,3 glycosidic bonds, which structure may be represented by the following formula:

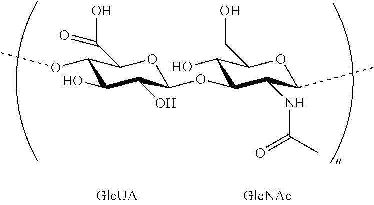

which shows a disaccharide unit, and wherein the number n of the repeating couples of units is such that the molecular weight of the polysaccharide is comprised between 50,000 and several millions of Dalton (Da).

In preferred embodiments of the invention, the average molecular weight of the hyaluronic acid (in the form of the corresponding sodium salt) is between 100,000 Da and 10,000,000 Da, and more preferably between 1,000,000 Da and 5,000,000 Da. In the most preferred embodiment, the average molecular weight of the hyaluronic acid (in the form of the corresponding sodium salt) is between 2,000,000 Da and 3,000,000 Da.

Hyaluronic acid can be isolated from various sources, for example, from human umbilical cord, cock's comb, or the connective tissue of vertebrates. Hyaluronic acid is also present in bacteria such as streptococci and may therefore also be obtained via fermentation processes.

Hyaluronic acid or a salt of hyaluronic acid can be used according to the present invention. Preferably, the salt is a pharmaceutically acceptable salt. Examples of pharmaceutically acceptable salts are alkali metal salts such as sodium or potassium salt or alkaline earth metal salts such as magnesium or calcium salt. In the most preferred embodiment, sodium hyaluronate is employed.

The ophthalmic composition of the present invention comprises an amount of hyaluronic acid or a pharmaceutically acceptable salt thereof ranging from 0.05% to 0.3% w/w, preferably from 0.1% to 0.25% w/w, and more preferably from 0.15% to 0.2% w/w.

Advantageously, the ophthalmic composition of the present invention comprises an amount of hyaluronic acid or a pharmaceutically acceptable salt thereof of about 0.15% w/w.

The ophthalmic composition of the present invention comprises glycogen and hyaluronic acid or a pharmaceutically acceptable salt thereof in a weight ratio ranging from about 5:1 to about 40:1, preferably from about 10:1 to about 30:1, more preferably from about 15:1 to about 25:1.

Advantageously, the ophthalmic composition of the present invention comprises glycogen and hyaluronic acid or a pharmaceutically acceptable salt thereof in a weight ratio of about 20:1.

Typically the ophthalmic composition according to the present invention has a viscosity of between 5 and 100 cP, preferably between 10 and 40 cP, and even more preferably between 15 and 30 cP.

Typically, the ophthalmic composition according to the present invention has an oncotic pressure of less than 5 mmHg. Preferably it has an oncotic pressure of less than 3 mmHg.

The ophthalmic composition according to the present invention may also contain other conventional ingredients such as one or more pharmaceutically acceptable buffering agents, preservatives, tonicity-adjusting agents, pH-adjusting agents, solubilizing agents, stabilizing agents, coloring agents, antioxidants, chelating agents, emollients, humectants and/or lubricants.

The buffering agents may include any weak conjugate acid-base pair suitable for maintaining a desirable pH range. Useful examples include, but are not limited to, bicarbonate buffer, acetate buffer, citrate buffer, phosphate buffer, borate buffer, or tromethamine (TRIS, 2-amino-2-hydroxymethyl-1,3-propanediol) buffer, and combination thereof. For example, combinations of monobasic phosphates, dibasic phosphates, and the like, or tromethamine and tromethamine hydrochloride can be used, and their quantities will be selected so as to regulate the pH of the ophthalmic composition according to the present invention between 5 and 9, preferably between 6 and 8. Preferably the buffer will be a phosphate buffer or a tromethamine buffer. Advantageously, the pH of the ophthalmic composition according to the present invention will be adjusted between 6.5 and 7.5.

The preservative may vary, and may include any compound or substance suitable for preventing microbial contamination in an ophthalmic formulation. Preservative agents are selected from the group comprising per-salts such as per-borates, per-carbonates and the like; alcohols, such as benzyl alcohol, chlorobutanol and the like; preservative agents containing quaternary ammonium salts such as benzalkonium chloride, benzalkonium bromide, polyquaternium;

guanidine-based preservatives including polyhexamethylene biguanide (PHMB), chlorhexidine and the like; mercury preservatives such as thimerosal, phenylmercuric acetate and phenylmercuric nitrate; metal chlorites, such as alkali metal and alkaline earth metal chlorites and the like; sorbic acid and ophthalmically acceptable salts such potassium sorbate and mixtures; oxidizing preservatives such as stabilized oxychloro complexes (e.g. Purite®). Purite® is a registered trademark of Allergan, Inc. The amount of preservative agents varies over a relatively wide range depending on the specific preservative agent employed. If the preservatives are not added to the ophthalmic solution, the ophthalmic solution can be used as single dose type eye drops, in which the ophthalmic solution is used off in one administration. Otherwise, the ophthalmic solution can be used as multi dose type eye drops included for example in a container provided with a filter attached to a nozzle of the container, for dispensing the eye drops, or included in an airless application system device.

Tonicity is adjusted by tonicity enhancing agents. Such agents may, for example, be of ionic and/or non-ionic type. Examples of ionic tonicity enhancers are alkali metal or earth metal halides, such as, for example, one or more of the following: calcium chloride, potassium chloride, sodium chloride, lithium chloride, potassium bromide, sodium bromide, sodium iodide, sodium phosphate, potassium phosphate, sodium and potassium sulfates, sodium and potassium bicarbonates, and boric acid. Non-ionic tonicity enhancing agents are, for example, urea, glycerol, sorbitol, mannitol, propylene glycol, dextrose or combinations thereof. Glycerin, sodium chloride and mannitol are the most preferred tonicity enhancing agents. The amount of tonicity agent may vary depending upon whether an isotonic, hypertonic, or hypotonic liquid is desired. The composition of the present invention generally has an osmolality in the range of 150-1500 mOsm/Kg, preferably in the range of 150-500 mOsm/Kg and most preferably in the range of 180-250 mOsm/Kg.

The ophthalmic composition according to the present invention can be prepared by dissolving the ingredients in an aqueous medium. Deionized water is the preferred aqueous medium, which can comprises minor amounts of other hydrophilic solvents, such as glycols and/or polyols. The composition can be prepared either by preparing a solution of one or more ingredients and then adding the remaining one or more ingredients, or by preparing two or more separate solutions, each comprising one or more ingredients, and then mixing such solutions all together.

In a preferred embodiment, the ophthalmic composition of the present invention is prepared by adding glycogen to a previously prepared aqueous solution of hyaluronic acid or a salt thereof, preferably sodium hyaluronate, and then adding the other conventional ingredients.

However, the exact order of addition of the conventional ingredients is not particularly relevant. As a non-limiting example, the buffer can be added after all of the active ingredients have been mixed rather than after the preparation of a solution containing only one of them. Usually, the adjustment of osmolarity and pH is the last step of the preparation, but intermediate additions of salt, acid and base, taking place between other steps of the invention, are contemplated by the inventors as being within the scope of the invention. The solution was finally sterilized by conventional methods, such as, by heating at high temperature, preferably between 50° C. and 80° C., more preferably from 60° to 80° C., for a period of time ranging from 30 minutes to several hours. Preferably, the sterile composition is obtained by heating at 70° C. for 1 hour and then filtering the solution through a 0.22 μm PES sterilization filter (as disclosed in US20110195925A1, herein incorporated by reference). More preferably, the sterile composition is obtained by filtering the solution through a 0.22 μm sterilization filter.

The following examples serve to illustrate the invention without however restricting it.

### EXAMPLE 1

### Preparation of Ophthalmic Solutions

A set of six ophthalmic solutions 1 to 6 was prepared by dissolving the components listed in the following Table 1 in the prescribed quantity of water at room temperature. After the complete solubilisation of all the ingredients, the solution was heated to 70° C. for 1 hour. Following the heat treatment step, the aqueous solution was filtered through a 0.22 μm PES sterilization filter to provide a sterilized solution.

TABLE 1

| | 1 (i) | 2 (i) | 3 (i) | 4 (i) | 5 (c) | 6 (c) |
|---|---|---|---|---|---|---|
| Sodium | 0.15 | 0.15 | 0.10 | 0.15 | 0.15 | — |
| Hyaluronate | | | | | | |
| Polglumyt | 3 | 3 | 3 | 3 | — | 3 |
| NaCl | 0.65 | — | — | 0.68 | 0.65 | 0.65 |
| Tromethamine | 0.091 | 0.091 | 0.091 | — | 0.091 | 0.091 |
| Mannitol | — | 3.5 | 3.5 | — | — | — |
| Na₂HPO₄*12 | — | — | — | 0.056 | — | — |
| H₂O | | | | | | |
| NaH₂PO₄ | — | — | — | 0.004 | — | — |
| HCl q.s. to pH | 7.2 | 7.2 | 7.2 | — | 7.2 | 7.2 |
| Water q.s. to | 100 | 100 | 100 | 100 | 100 | 100 |
| mL | | | | | | |

The six ophthalmic solutions 1 to 6 prepared as described above had the properties summarized in the following Table 2.

TABLE 2

| | 1 (i) | 2 (i) | 3 (i) | 4 (i) | 5 (c) | 6 (c) |
|---|---|---|---|---|---|---|
| pH | 7.2 | 7.2 | 7.2 | 7.2 | 7.2 | 7.2 |
| Osmolality (mOsm/Kg) | 234 | 232 | 231 | 235 | 234 | 233 |
| Viscosity (cP) | 16 | 55 | 17 | 18 | 20 | 2 |
| Sterility | Yes | Yes | Yes | Yes | Yes | Yes |

Osmolality was determined using a Knauer Automatic Osmometer apparatus. Viscosity was determined using a Bohlin Gemini 150 rheometer at a stress of 0.5 Pa and at 25° C.

### EXAMPLE 2

### Determination of Mucoadhesive Properties

Mucoadhesion can be defined as the state in which two materials, at least one of which is a biological substrate such as mucin, are maintained together for a prolonged time by means of interfacial forces. Mucous membranes of human body, including nasal, ocular, buccal, vaginal, and rectal membranes, are characterized by an epithelial layer whose surface is covered by mucus. The mucus contains glycoproteins, the most important of which is mucin. Mucin is involved in the mechanism of adhesion by establishing interactions with macromolecules contained in mucoadhesive formulations. A rheological test based on the measurement of viscosity is a simple in vitro method used to measure the formulation-mucin interactions. From such a test, it is possible to obtain the mucoadhesion force by monitoring the viscosimetric changes of the system constituted by the mixture of the formulation under examination and mucin compared with the sum of the systems only constituted by the formulation and mucin, respectively (Hassan, E. E., Gallo, J. M., “A simple rheological method for the in vitro assessment of mucin-polymer bioadhesive bond strength”, Pharm. Res., v. 7, n. 5, p. 491-495, 1990).

Gastric porcine mucin (type II) (Sigma-Aldrich, Milano, Italy) was suspended at 4% w/w and 8% w/w in a simulated tear fluid containing 6.8 g/l NaCl, 2.2 g/l NaHCO₃, 0.084 g/l CaCl₂ 2 H₂O, 1.4 g/l KCl and adjusted to pH 7.4 with HCl 1N.

The viscosity measurements were performed by means of a rotational rheometer (Rheostress 600, Haake, Enco, Italy), equipped with a cone plate combination (CP1/60).

The test was performed with ophthalmic solutions 1, 2 and 4 of the invention. For each rheological test the following samples were prepared and tested:
mucin dispersion at 4% w/w in simulated tear fluid (sample A);
the ophthalmic solution of the present invention mixed with simulated tear fluid at a 1:1 weight ratio (sample B);
the ophthalmic solution of the present invention mixed with 8% mucin dispersion in simulated tear fluid at a 1:1 weight ratio (sample C).

Each sample was subjected to viscosity measurements at 32° C. The interactions between mucin and the composition of the present invention were quantified by means of the bioadhesion viscosity component Δη at a range of shear rate (10-100 1/s), as follow:

Δη=η_{C}−(η_{B}+η_{A})

where: η_{C} is the viscosity of sample C (Pa·S); η_{B} is the viscosity of sample B (Pa·S), η_{A} is the viscosity of sample A (Pa·S), and Δη is the bioadhesion viscosity component.

An increase in the viscosity of the mixture of the composition of the present invention with mucin (sample C) compared to the sum of the viscosity of the composition of the present invention (sample B) and mucin (sample A) solutions alone shows a positive bioadhesion viscosity component (Δη>0) and therefore mucoadhesive properties of the composition.

A positive bioadhesion viscosity component represents a growth of the mixture viscosity, which occurs when the composition of the present invention is mixed with mucin dispersion, and which depends on the interactions between the chains of the macromolecular species.

In other words, a value higher than zero of the component Δη represents the extra contribution to viscosity by the interaction of the mucin with the composition of the present invention, compared to the value expected on the basis of a simple addition of the viscosity contribution given by the mucin and the composition of the present invention, taken separately.

The results of each rheological test with ophthalmic solutions 1, 2 and 4 of the invention at different shear rate are summarized in the following Table 3.

TABLE 3

| Shear rate | Δη 1 (i) | | Δη 2 (i) | | Δη 4 (i) | |
|---|---|---|---|---|---|---|
| (1/s) | (mPa*s) | SD 1 (i) | (mPa*s) | SD 2 (i) | (mPa*s) | SD 4 (i) |
| 10 | 12.58 | 2.05 | 17.19 | 4.00 | 20.55 | 2.50 |
| 15 | 12.56 | 1.55 | 14.90 | 2.45 | 19.47 | 2.51 |
| 20 | 12.29 | 1.10 | 14.26 | 1.32 | 18.63 | 1.76 |
| 25 | 12.25 | 0.90 | 13.64 | 0.62 | 17.97 | 2.01 |
| 30 | 11.72 | 0.85 | 13.33 | 1.00 | 17.07 | 1.62 |
| 40 | 10.95 | 0.825 | 11.93 | 0.51 | 15.51 | 1.50 |
| 50 | 10.37 | 0.52 | 11.09 | 0.10 | 14.59 | 1.05 |
| 60 | 9.95 | 0.40 | 10.44 | 0.05 | 14.14 | 0.76 |
| 70 | 9.54 | 0.26 | 9.85 | 0.05 | 13.69 | 0.37 |
| 80 | 9.25 | 0.10 | 9.42 | 0.12 | 13.22 | 0.09 |
| 90 | 9.04 | 0.06 | 9.06 | 0.12 | 12.88 | 0.05 |
| 100 | 8.76 | 0.02 | 8.73 | 0.26 | 12.55 | 0.05 |

SD: Standard Deviation

### EXAMPLE 3

### In Vitro Models for Studying the Symptoms of the Ocular Discomfort

The synergistic effect on therapeutic effectiveness provided by the combination of sodium hyaluronate and glycogen is described by two different in vitro models: a model for studying the symptoms of the dry eye and a model for studying the surface epithelial damage.

The first model is a 3D human corneal dryness and hyper-osmolarity model. The parameters monitored demonstrated the synergistic effect of sodium hyaluronate and glycogen in reducing inflammatory parameters, and in protecting the eye from an excessive matrix degradation.

The model for studying the surface epithelial damage is an in vitro model used to monitor human corneal epithelium response to mechanical injuries. This model was used to demonstrate the synergistic effect of the combination of the present invention in promoting corneal re-epithelization consequent to surface epithelial damage.

Both models employed the 3D reconstructed human corneal epithelium (HCE), supplied by SkinEthic® Laboratories (Nice, France). HCE is a model consisting of immortalized HCE cells with an overall morphology similar to that of human corneal epithelium.

### EXAMPLE 3.1

### Model for Studying the Symptoms of the Dry Eye

In this example, the HCE has been used to set up a model of human corneal dryness and hyper-osmolarity (HYP-DRY HCE).

HCE tissues were placed under controlled environmental conditions to mimic dryness (<40% relative humidity, T°>37° C. in the presence of sorbitol 0.6 M in the medium) for 16 h. At the end of the stress period, the samples were treated with the products (30 μL) for 24 h and the tissues were investigated for different parameters (mRNA expression, histological and ultrastructural analysis). The test was performed with ophthalmic solution 1 of the present invention and comparative ophthalmic solution 5 and 6.

### EXAMPLE 3.1a

### Transcriptional Analysis

Total mRNA extracted from HCE has been analyzed by transcriptional analysis (Real Time PCR) to quantify the expression of Matrix Metallopeptidase-9 (MMP-9) and Integrin-β1 (ITG-β1).

MMP-9 is the most important gelatinase present on the ocular surface. This enzyme lyses a variety of different substrates including components of the corneal epithelial basement membrane and tight junction proteins that maintain corneal epithelial barrier function. High levels of MMP-9 are dosed in tear fluids of patients with dry eye. Tear MMP-9 activity levels correlated positively with the severity of corneal disease. Increased expression of MMP-9 correlated to increased ocular surface inflammation.

ITG-β1 is a member of the large family of integrins. Integrins are key components for migration and activation of immune cells into the ocular surface of patients with dry eye. It has been demonstrated that ITG-β1 can serve as a target for treatment of inflammatory disorders. Topical application of an α4β1-integrin antagonist lead to disease remission; blockade of α4β1 decreased dry eye symptoms and inflammation. Increase of ITG-β1 is a signal of dry eye symptoms and inflammation indicating the activation of immune cells into the ocular surface.

The results shown in the following Table 4 were expressed as Relative Quantification (RQ) indicating the fold change in the expression compared to the calibrator (non-treated HCE tissue).

TABLE 4

| | MMP-9 | ITG-β1 |
|---|---|---|
| HCE | 1 | 1 |
| HYP-DRY HCE | 2.82* | 5.39* |
| Solution 1 | 2.08* | 4.8* |
| Solution 5 | 2.71* | 11.37* |
| Solution 6 | 2.40* | 6.28* |

*The value is considered up regulated when RQ > 2 or down regulated when RQ < 0.5 compared to non-treated HCE (RQ = 1)

The results clearly shown that comparison solution 5 did not change levels of MMP-9 with respect to HYP-DRY HCE (2.71 vs 2.82), and comparison solution 6 is only able to slightly decrease levels of MMP-9 with respect to HYP-DRY HCE (2.4 vs 2.82), while solution 1 of the invention induced the highest decrease of MMP-9 expression, indicating a protection from excessive matrix degradation (2.08 vs 2.82).

The results further clearly shown that comparison solutions 5 and 6 induced an overexpression of ITG-131, while solution 1 of the invention produced the lowest expression compared to positive control (HYP-DRY HCE). Reduction of ITG-131 is a positive signal for decreasing dry eye symptoms and inflammation.

### EXAMPLE 3.1b

### Ultrastructural Analysis

Ultrastructural analysis was performed using Scanning Electron Microscopy (SEM). Samples were observed with a SEM Zeiss Sigma Electron Microscope. Magnification of 2000× has been performed. A score attributed to the corneal epithelial was based on the quality assessment of corneal smoothness: 0 (standard: smoothest surface), 1 (slight), 2 (strong) and 3 (severe: surface ruffling).

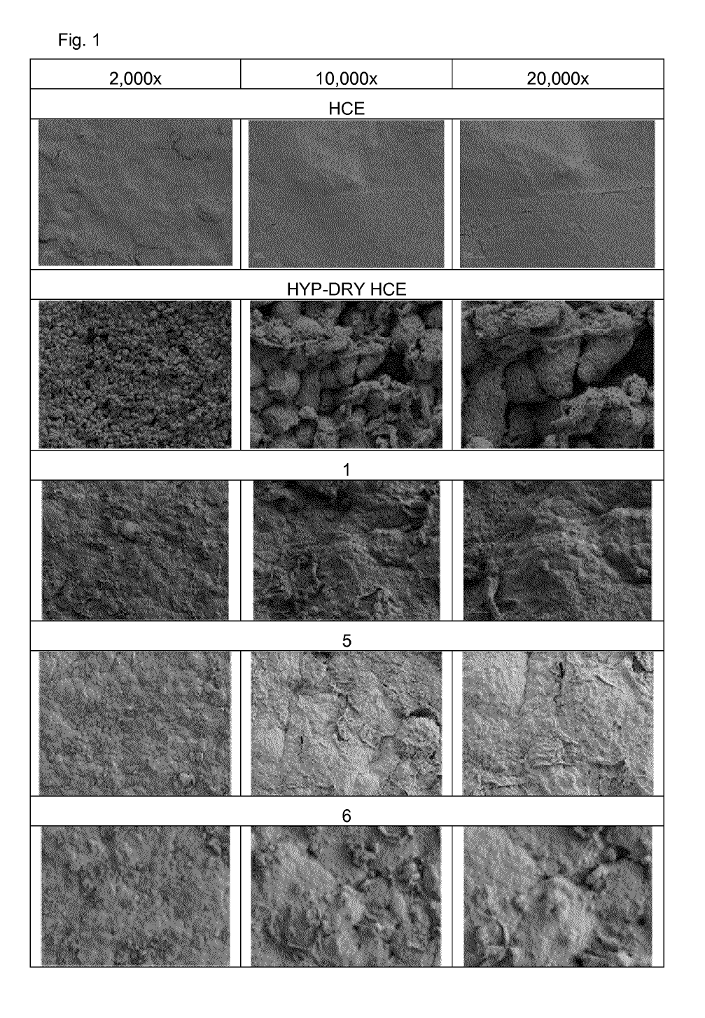

The results are summarized in the following Table 5 and in FIG. 1.

TABLE 5

| | SEM Score |
|---|---|
| HCE | 0 |
| HYP-DRY HCE | 3 |
| Solution 1 | 1 |
| Solution 5 | 0/1 |
| Solution 6 | 1 |

### EXAMPLE 3.2

### Model for Studying the Surface Epithelial Damage

In this example, the HCE has been used to set up a model of human corneal wound healing. HCEs were injured with 4 symmetrical injuries on epithelium surface and 1 h after injury tissues have been treated with the products (30 μL) for 24 h and 72 h. At the end of the treatment the tissues were investigated for different parameters (mRNA expression, immunofluorescence, histological and ultrastructural analysis). The test was performed with ophthalmic solution 1 of the present invention and comparative ophthalmic solution 5 and 6.

### EXAMPLE 3.2a

### Transcriptional Analysis

Total mRNA extracted from HCE has been analyzed by transcriptional analysis (Real Time PCR) to quantify the expression of Matrix Metallopeptidase-1 (MMP-1).

Matrix metalloproteinases (MMPs) are a group of zinc-dependent proteinases whose substrates include most components of the extracellular matrix and basement membrane. After injury, and in response to the release of cytokines, several MMPs in the cornea are upregulated by transcription or activation. MMP-1 is a key mediator of epithelial migration. Studies of ex vivo wounded human corneal tissue confirmed the presence of MMP-1 in the leading corneal epithelial cells during re-epithelialization over stroma.

The results shown in the following Table 6 were expressed as Relative Quantification (RQ) indicating the fold change in the expression compared to the calibrator (non-treated HCE tissue).

TABLE 6

| | MMP-1 at |
|---|---|
| | 24 hours |
| HCE | 1 |
| Injured HCE | 8.89* |
| Solution 1 | 2.73* |
| Solution 5 | 1.68* |
| Solution 6 | 1.84* |

*The value is considered up regulated when RQ > 2 or down regulated when RQ < 0.5 compared to non-treated HCE (RQ = 1)

In the injured tissue, MMP-1 was shown to be upregulated at 24 h demonstrating a first positive reaction of cells to re-epithelialization and matrix remodeling.

The solutions 5 and 6 strongly decreased the level of MMP-1 at 24 h, so indicating a reduced re-epithelialization process and matrix remodeling. The higher level of MMP-1, promoted by solution 1 of the invention, is a positive sign for re-epithelialization and matrix remodeling.

### EXAMPLE 3.2b

### Histological Analysis

At the end of the exposures tissues were fixed in buffered 10% formalin and included in paraffin blocks in order to obtain sections of 5 μm. Slides were stained with hematoxylin and eosin and analyzed under a light microscopy (20×). The progression of healing was assessed to compare the healing status in the control tissues. It has been used a classification based on the healing rate (good>fair>poor).

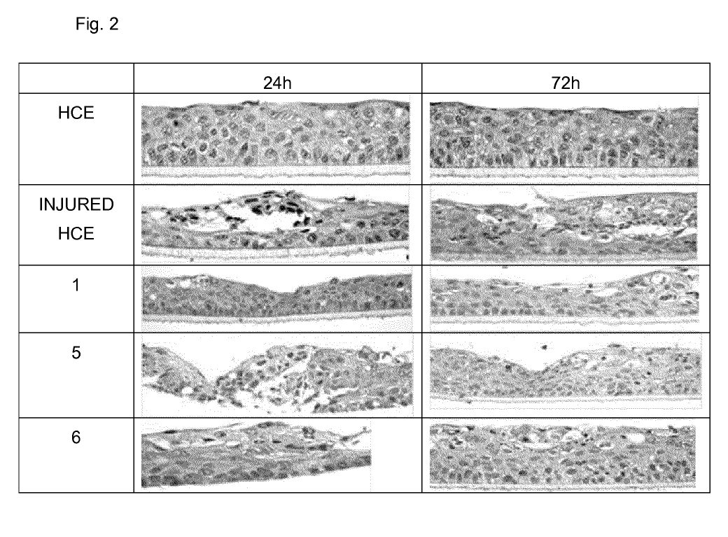

The results are summarized in the following Table 7 and in FIG. 2.

TABLE 7

| | Healing rate |
|---|---|
| HCE | — |
| Injured HCE | Poor |
| Solution 1 | Good |
| Solution 5 | Fair |
| Solution 6 | Fair |

### EXAMPLE 3.2c

### Ultrastructural Analysis

Ultrastructural analysis was performed using Scanning Electron Microscopy (SEM). Samples were observed with a SEM Zeiss Sigma Electron Microscope. Magnification of 2000× has been performed. A score attributed to the wound corneal epithelial was based on the characteristic changes in migrating epithelial cells: 0 (standard: absence of wounded surface), 1 (maintenance and regeneration or corneal epithelial cell layer), 2 (not complete regeneration) and 3 (absence of re-epithelialization).

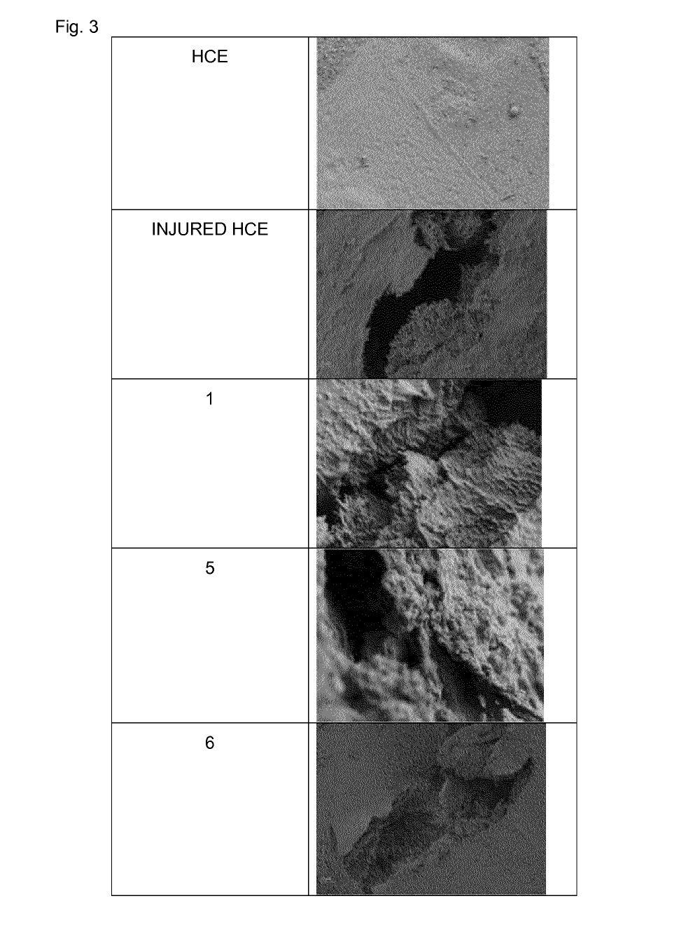

The results are summarized in the following Table 8 and in FIG. 3.

TABLE 8

| | SEM Score |
|---|---|
| HCE | — |
| Injured HCE | 2-3 |
| Solution 1 | 1 |
| Solution 5 | 3 |
| Solution 6 | 1 |

1. An ophthalmic composition comprising a synergistic combination of glycogen and hyaluronic acid or a pharmaceutically acceptable salt thereof, and at least one pharmaceutical acceptable excipient, wherein said composition comprises an amount of said glycogen ranging from 1% to 6% w/w and an amount of said hyaluronic acid or a pharmaceutically acceptable salt thereof ranging from 0.05% to 0.3% w/w.

2. The ophthalmic composition according to claim 1, wherein said composition comprises an amount of said glycogen ranging from 2% to 5% w/w, preferably from 3% to 4% w/w.

3. The ophthalmic composition according to claim 2, wherein said composition comprises an amount of said glycogen of about 3% w/w.

4. The ophthalmic composition according to claim 1, wherein said composition comprises an amount of said hyaluronic acid or pharmaceutically acceptable salt thereof ranging from 0.1% to 0.25% w/w, preferably from 0.15% to 0.2% w/w.

5. The ophthalmic composition according to claim 4, wherein said composition comprises an amount of said hyaluronic acid or pharmaceutically acceptable salt thereof of about 0.15% w/w.

6. The ophthalmic composition according to claim 1, wherein said composition comprises glycogen and hyaluronic acid or a pharmaceutically acceptable salt thereof in a weight ratio ranging from about 5:1 to about 40:1, preferably from about 10:1 to about 30:1, more preferably from about 15:1 to about 25:1.

7. The ophthalmic composition according to claim 6, wherein said composition comprises glycogen and hyaluronic acid or a pharmaceutically acceptable salt thereof in a weight ratio of about 20:1.

8. The ophthalmic composition according to claim 1, wherein said composition has a viscosity of between 5 and 100 cP, preferably between 10 and 40 cP, and more preferably between 15 and 30 cP.

9. The ophthalmic composition according to claim 1, wherein said composition has an oncotic pressure of less than 5 mmHg, preferably of less than 3 mmHg.

10. The ophthalmic composition according to claim 1, wherein said composition has an osmolality in the range of 150-1500 mOsm/Kg, preferably in the range of 150-500 mOsm/Kg, and most preferably in the range of 180-250 mOsm/Kg.

11. An ophthalmic composition for use in the treatment of dry eye syndrome comprising a synergistic combination of glycogen and hyaluronic acid or a pharmaceutically acceptable salt thereof, and at least one pharmaceutical acceptable excipient.

12. A method for the treatment of dry eye syndrome, wherein the method consists in applying a therapeutically effective amount of an ophthalmic composition comprising a synergistic combination of glycogen and hyaluronic acid or a pharmaceutically acceptable salt thereof, and at least one pharmaceutical acceptable excipient to a patient in need thereof.

13. A process for the preparation of the ophthalmic composition as defined in claim 1 comprising the steps of dissolving said glycogen and hyaluronic acid or a pharmaceutically acceptable salt thereof in an aqueous medium, adding said at least one pharmaceutical acceptable excipient, adjusting the concentration of said glycogen in the range from 1% to 6% w/w and the concentration of said hyaluronic acid or a pharmaceutically acceptable salt thereof in the range from 0.05% to 0.3% w/w, adjusting the pH of said ophthalmic composition to a value ranging from 6 to 8, adjusting the osmolality of said ophthalmic composition to a value ranging from 150 to 1,500 mOsm/Kg, and sterilizing said ophthalmic composition.
